(19) United States
(12) Patent Application Publication
(10) Pub. No.: US 2003/0187374 A1
(43) Pub. Date: Oct. 2, 2003

(54) Nose shaping device

(76) Inventor: Masaki Nishioka, Kanagawa (JP)

Correspondence Address: Yusuke Takeuchi, Kaneska & Takeuchi, 1423 Powhatan Street, Alexandria, VA, 22314, US

(21) Appl. No.: 10/381,281

(22) PCT Filed: Sep. 26, 2001
(86) PCT No.: PCT/JP01/08343

(30) Foreign Application Priority Data
Oct. 3, 2000 (JP) 2000-303763
May 29, 2001 (JP) 2001-160639

(51) Int. Cl. A61F 5/08

(52) U.S. Cl. 602/5

(57) ABSTRACT

A nose ridge reshaping device allowing plastically reshaping of a nose with ease by merely sticking it on a face, correcting the ridge of a nose to an attractive feeling, and reducing or making inconspicuous the wrinkles between the brows that are easy to be produced on the face. First, an adhesive fixing piece (20) is stuck on the face area ranging from between both brows to the upper side thereof to fix the entire nose ridge reshaping device (10) onto the face. Next, a nose ridge reshaping piece (30) extending from the lower end of the adhesive fixing piece (20) directly downward is stuck to the nose, a natural curve being formed along the original ridge of the nose with the cross sectional surface thereof being kept substantially linear. Then, the nose ridge reshaping piece (30) is bent in the crosswise direction such that the cross sectional surface thereof pinches the ridge of the nose, whereby the nose ridge reshaping piece (30) is deformed into the state of being extended linearly in the longitudinal direction and the ridge of the nose is held in the state of being raised forward, which provides a clear straightforwardly extending ridge of nose.

### FIELD OF THE INVENTION

The present invention relates to a nose ridge reshaping device allowing reshaping the ridge of a nose into a clear straightforwardly extending shape by sticking it on a face.

### BACKGROUND

For both men and women, a clear straightforwardly extending ridge of nose has been a major feature of a good-looking face. Nose ridge shape, however, varies greatly from person to person and is rather unsatisfactory for most people.

Although those who have a great desire for a face with an extending ridge of nose may choose to receive cosmetic surgery, it has been an uncommon measure because it requires surgical operation and is usually very expensive.

Another major concern about facial looks has been the presence of wrinkles. When reaching a certain age, people tend to have wrinkles in such areas as between the brows because of the lost elasticity of the skin or some other factor. Eliminating or making inconspicuous such wrinkles has been another major cosmetic demand of women.

However, because there has been no cosmetic device capable of easily reshaping the ridge of a nose into a clear straightforwardly extending shape, as stated above, it has been impossible to easily satisfy the demands of those interested in beauty. As is the case with nose ridge reshaping, there has been no means for effectively correcting wrinkles.

### SUMMARY OF THE INVENTION

Being developed in consideration of the conventional problems as stated above, the purpose of the present invention is to provide a nose ridge reshaping device allowing plastically reshaping the ridge of a nose with ease by merely sticking it on a face while sleeping at night, for example, correcting the ridge of a nose to an attractive feeling and reducing or making inconspicuous the wrinkles between the brows that are easy to produce on the face.

The subject matters of the present invention to achieve the above purpose are disclosed in the following items:

[1] A nose ridge reshaping device allowing reshaping of the ridge of a nose into a clear straightforwardly extending shape by sticking it on a face, comprising:

an adhesive fixing piece to be stuck on the area ranging from between both brows to the forehead; and

a nose ridge reshaping piece extending from the adhesive fixing piece downward with a defined width that is plastic and can be curved along the ridge of the nose,

wherein said nose ridge reshaping piece can be deformed in the longitudinal direction, with the cross sectional surface thereof being held substantially linearly, and is stuck from said adhesive fixing piece to the tip of the nose along the original ridge of the nose and then bent in the crosswise direction such that the cross sectional surface thereof pinches the ridge of the nose, whereby the longitudinal sectional surface thereof is held in the state of being extended substantially linearly, with the ridge of the nose ranging from the upper end of the nose ridge reshaping piece to the tip of the nose being raised forward.

[2] A nose ridge reshaping device allowing reshaping of the ridge of a nose into a clear straightforwardly extending shape by sticking it on a face, comprising:

an adhesive fixing piece to be stuck on the area ranging from between both brows to the forehead; and

a nose ridge reshaping piece extending from the adhesive fixing piece downward with a defined width that is plastic and can be curved along the ridge of the nose,

wherein said nose ridge reshaping piece is provided with a pair of fold grooves extending in the longitudinal direction and substantially parallel to each other,

said nose ridge reshaping piece being stuck from said adhesive fixing piece to the tip of the nose along the original ridge of the nose, being deformable in the longitudinal direction, with the cross sectional surface thereof being held substantially linearly, and then bent at said pair of fold grooves in the crosswise direction such that the cross sectional surface thereof pinches the ridge of the nose, whereby the longitudinal sectional surface is held in the state of being extended substantially linearly, with the ridge of the nose being raised forward by the nose ridge reshaping piece.

[3] A nose ridge reshaping device allowing reshaping of the ridge of a nose into a clear straightforwardly extending shape by sticking it on a face, comprising:

an adhesive fixing piece to be stuck on the area ranging from between both brows to the forehead; and a nose ridge reshaping piece extending from the adhesive fixing piece downward with a defined width that is plastic and can be curved along the ridge of the nose,

wherein said nose ridge reshaping piece is provided with a pair of fold grooves extending in the longitudinal direction and forming curves that approach each other at substantially middle points,

said nose ridge reshaping piece being stuck from said adhesive fixing piece to the tip of the nose along the original ridge of the nose, being deformable in the longitudinal direction, with the cross sectional surface thereof being held substantially linearly, and then bent at said pair of fold grooves in the crosswise direction such that the cross sectional surface thereof pinches the ridge of the nose, whereby the longitudinal sectional surface thereof is held in the state of being extended forward with a slight curve, with the ridge of the nose being raised forward by the nose ridge reshaping piece.

[4] The nose ridge reshaping device according to [1], [2] or [3], wherein each of said adhesive fixing piece and nose ridge reshaping piece comprises a plastic substrate sheet which back is coated with an adhesive.

[5] The nose ridge reshaping device according to [1], [2] or [3], wherein each of said adhesive fixing piece and nose ridge reshaping piece comprises a plastic substrate sheet which back is laminated with a pressure sensitive adhesive sheet, the pressure sensitive adhesive sheet being replaceable with a new pressure sensitive adhesive sheet.

[6] The nose ridge reshaping device according to [1], [2], [3], [4] or [5], further comprising:

a holder that can be attached to the bent nose ridge reshaping piece so as to pinch it at an intermediate point when said nose ridge reshaping piece is bent in the crosswise direction, in order to hold said bent geometry.

[7] The nose ridge reshaping device according to [1], [2], [3], [4], [5] or [6], wherein the lower end of said nose ridge reshaping piece is provided with a hook terminal that can be stuck to the tip of the nose so as to enclose it.

[8] The nose ridge reshaping device according to [7], further comprising:

a support that is interposed between said hook terminal and the bottom of the nose such that the hook terminal is raised forward when the hook terminal encloses the tip of the nose.

[9] The nose ridge reshaping device according to [1], [2], [3], [4], [5], [6], [7] or [8], wherein a fold origin of the nose ridge reshaping piece is provided on the border between the upper end of said nose ridge reshaping piece and the lower end of said adhesive fixing piece.

[10] The nose ridge reshaping device according to [1], [2], [3], [4], [5], [6], [7], [8] or [9], wherein at least a part of said adhesive fixing piece and said nose ridge reshaping piece are formed separately and combined when used.

[11] The nose ridge reshaping device according to [1], [2], [3], [4], [5], [6], [7], [8], [9] or [10], wherein said adhesive fixing piece is formed so as to extend from between both brows laterally along the upper edges of the brows and is stuck so as to stretch and correct the wrinkles in the area covered by the adhesive fixing piece.

[12] The nose ridge reshaping device according to [11], wherein said adhesive fixing piece is formed so as to extend laterally in a curve to fit the forehead.

When the nose ridge reshaping device according to the present invention is used, the adhesive fixing piece is first stuck on the face area ranging from between both brows to the upper side thereof to fix the entire nose ridge reshaping device onto the face.

Next, the nose ridge reshaping piece extending from the lower end of the adhesive fixing piece downward along the ridge of the nose is securely stuck from the upper end thereof, which connects to said adhesive fixing piece, to the tip of the nose, with the cross sectional surface thereof being held substantially linearly and a natural curve being created along the original ridge of the nose.

Then, the nose ridge reshaping piece is bent in the crosswise direction such that the cross sectional surface thereof pinches the ridge of the nose. As a result, the nose ridge reshaping piece is plastically deformed from the state of being curved along the ridge of the nose into the state of being extended linearly in the longitudinal direction. Because the ridge of the nose to which the nose ridge reshaping piece is stuck is held in the state of being raised forward, a clear straightforwardly extending ridge of nose can be provided.

According to another nose ridge reshaping device, the nose ridge reshaping piece is provided with a pair of fold grooves extending in the longitudinal direction and substantially parallel to each other. Therefore, the nose ridge reshaping piece can be easily bent at the fold grooves such that the cross sectional surface thereof forms a substantially Π shape when the nose ridge reshaping piece is bent in the crosswise direction such that the cross sectional surface thereof pinches the ridge of the nose.

In still another nose ridge reshaping device, the nose ridge reshaping piece may be provided with a pair of fold grooves extending in the longitudinal direction and forming curves that approach each other at substantially middle points. According to such curved fold grooves, when the nose ridge reshaping piece is bent in the crosswise direction at said fold grooves so as to pinch the ridge of the nose, the nose ridge reshaping piece is plastically deformed into and held in the state of raising the ridge of the nose forward, with the longitudinal sectional surface thereof being extended forward with a slight curve.

Each of said adhesive fixing piece and nose ridge reshaping piece may comprise a substrate sheet which back is coated with an adhesive, and can be manufactured at a relatively low cost.

If a pressure sensitive adhesive sheet is used in place of the adhesive and it is possible to replace the pressure sensitive adhesive sheet alone with a new pressure sensitive adhesive sheet, the substrate sheets of the adhesive fixing piece and the nose ridge reshaping piece can be repeatedly used by merely replacing the pressure sensitive adhesive sheet whose adhesion capability has been decreased, which can lead to a reduction in cost.

If a holder is attached to the bent nose ridge reshaping piece so as to pinch it at an intermediate point when said nose ridge reshaping piece is bent in the crosswise direction, the bent form can be secured for a long period of time.

If the lower end of said nose ridge reshaping piece is provided with a hook terminal that can be stuck to the tip of the nose so as to enclose it, the lower end of the nose ridge reshaping piece can be firmly fixed to the tip of the nose.

If a support is interposed between the hook terminal and the bottom of the nose such that the hook terminal is raised forward when the hook terminal encloses the tip of the nose, it prevents the tip of the nose from being pressed and crushed by said nose ridge reshaping piece in reaction to the longitudinal extension of said nose ridge reshaping piece, allowing the entire nose, including the tip, to be raised forward securely.

If a fold origin of the nose ridge reshaping piece is provided on the border between the upper end of said nose ridge reshaping piece and the lower end of said adhesive fixing piece, it prevents said adhesive fixing piece from being subjected to an undesired peeling force when the cross sectional surface of the nose ridge reshaping piece is bent in the crosswise direction and the nose ridge reshaping piece is extended in the longitudinal direction.

At least a part of said adhesive fixing piece and the nose ridge reshaping piece may be originally formed integrally or may be formed separately and combined when used.

If said adhesive fixing piece is formed so as to extend from between both brows laterally along the upper edges of the brows and is stuck to the face so as to stretch and correct the wrinkles in the area covered by the adhesive fixing piece, wrinkles can be reduced or made inconspicuous, at the same time.

In this case, if the adhesive fixing piece is formed so as to extend laterally in a curve to fit the forehead, it prevents the skin in the vicinity of hairline from being pulled excessively, eliminating the possibility of inhibiting hair growth.

### BRIEF DESCRIPTION OF THE DRAWINGS

FIG. 1 is a plan view showing a nose ridge reshaping device of a first embodiment of the present invention;

FIG. 2 is a sectional view taken along line II-II of FIG. 1;

FIG. 3 is a perspective view showing the nose ridge reshaping device of the first embodiment of the present invention when stuck on the face and in use;

FIG. 4(A) and FIG. 4(B) are side views describing how to use the nose ridge reshaping device of the first embodiment of the present invention;

FIG. 5(A) and FIG. 5(B) are sectional views describing how to use the nose ridge reshaping device of the first embodiment of the present invention;

FIG. 6(A) and FIG. 6(B) are perspective views describing the hook terminal in use of the nose ridge reshaping piece in the nose ridge reshaping device of the first embodiment of the present invention;

FIG. 7 is a functional explanatory view of the nose ridge reshaping piece in the nose ridge reshaping device of the first embodiment of the present invention;

FIG. 8 is a plan view showing the nose ridge reshaping device of a second embodiment of the present invention;

FIG. 9 is a sectional view taken along line IX-IX of FIG. 8;

FIG. 10(A) and FIG. 10(B) are sectional views describing how to use the nose ridge reshaping device of the second embodiment of the present invention;

FIG. 11 is a plan view showing the nose ridge reshaping device of a third embodiment of the present invention;

FIG. 12 is a functional explanatory view of the nose ridge reshaping piece in the nose ridge reshaping device of the third embodiment of the present invention;

FIG. 13 is a perspective view showing a holder of the nose ridge reshaping device of a fourth embodiment of the present invention;

FIG. 14 is a functional explanatory view of the holder of the nose ridge reshaping device of the fourth embodiment of the present invention;

FIG. 15 is a perspective view showing a support of the nose ridge reshaping device of a fifth embodiment of the present invention; and

FIG. 16 is a functional explanatory view of the support of the nose ridge reshaping device of the fifth embodiment of the present invention.

### DESCRIPTION OF THE PREFERRED EMBODIMENTS

Exemplary embodiments of the present invention will now be described with reference to the drawings.

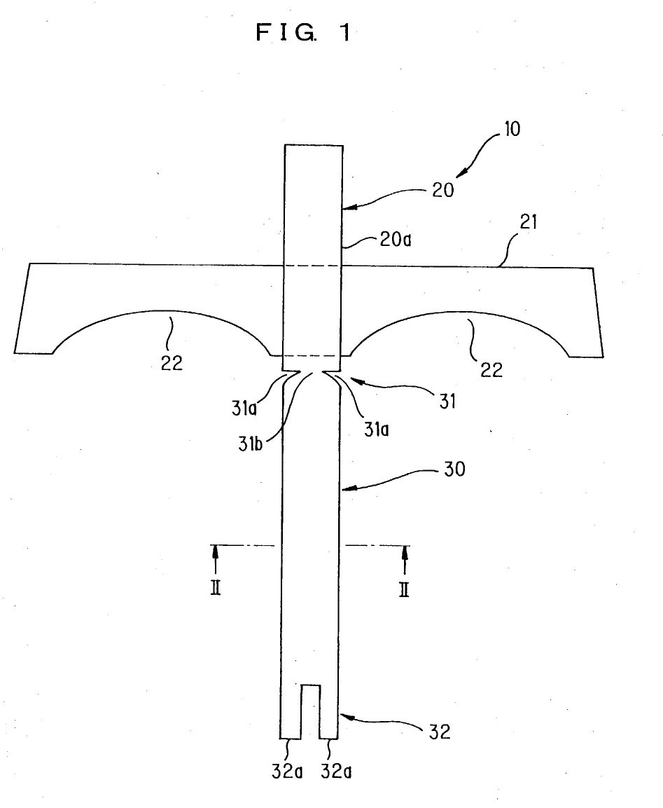

FIG. 1 to FIG. 7 show a first embodiment of the present invention.

A nose ridge reshaping device 10 according to the first embodiment allows reshaping the ridge of a nose into a clear straightforwardly extending one by merely sticking it on a face, and making inconspicuous or correcting the wrinkles between the brows, at the same time.

As shown in FIG. 1 and FIG. 3, the nose ridge reshaping device 10 comprises an adhesive fixing piece 20 to be stuck on the area ranging from between both brows to the forehead and a nose ridge reshaping piece 30 extending, with a defined width, from a substantially middle portion of the lower end of the adhesive fixing piece 20 directly downward along the ridge of the nose, having plasticity, and capable of being curved along the ridge of the nose.

In this embodiment, the adhesive fixing piece 20 comprises a main body 20a and a forehead-wrinkle eliminating member 21. The main body 20a of the adhesive fixing piece 20 is formed integrally with the nose ridge reshaping piece 30, while the forehead-wrinkle eliminating member 21 is formed separately from the main body 20a and the nose ridge reshaping piece 30.

In operation, the main body 20a of the adhesive fixing piece 20 and the nose ridge reshaping piece 30 cross the forehead-wrinkle eliminating member 21 at substantially right angles, as shown in FIG. 1. However, the forehead-wrinkle eliminating member 21 may be formed integrally with the nose ridge reshaping piece 30. As described later, it should be noted that the specific forms of the adhesive fixing piece 20 and the nose ridge reshaping piece 30 are not limited to those provided in the drawings.

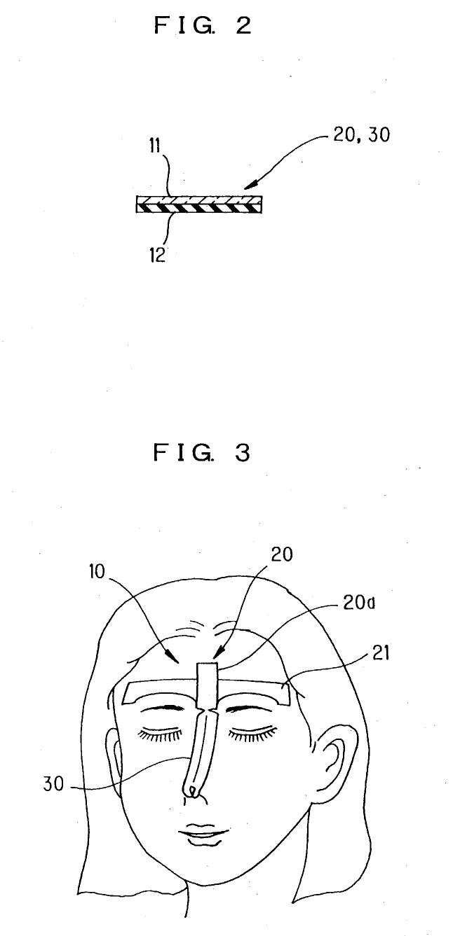

As shown in FIG. 2, each of the adhesive fixing piece 20 and the nose ridge reshaping, piece 30 comprises a plastic substrate sheet 11, which is cut to shape and whose back is coated with an adhesive 12. The substrate sheet 11 used may be of any material that has plasticity as well as moderate flexibility and hardness, and offers no risk of causing damage to the skin.

Specifically, a sheet made of a soft synthetic resin, such as vinyl chloride, is suitable for use as substrate sheet 11. Because the nose ridge reshaping piece 30 requires higher hardness and plasticity than those of the adhesive fixing piece 20, the substrate sheet 11 for the nose ridge reshaping piece 30 may be made of a thin metal film coated with plastic, or a shape-memory material, which can return to the original shape thereof after being released from the deformation.

The adhesive 12 directly adheres to the skin, and thus requires higher safety, being composed of such substances as rubber and tackifier. The component rubber may be synthetic or natural one, and the tackifier may be terpene resin, gum rosin, or the like. Further, in order to add the so-called packing effect to the adhesive 12, the adhesive may be mixed with a nourishing cream or the like in an amount that will not lessen the adhesion capability.

If a large number of pores are provided over the entire surfaces of the adhesive fixing piece 20 and the nose ridge reshaping piece 30, each of which comprises the substrate sheet 11 coated with the adhesive 12, air permeability to the skin is assured and undesired inhibition of cutaneous respiration is prevented. Preferably, the adhesive 12 has a backing material (not shown) that covers the adhesive 12 to retain adhesion capability and is peeled off before use. A commercially available medical tape or the like may be used as the forehead-wrinkle eliminating member 21.

The adhesive fixing piece 20 is stuck on the face area ranging from between both brows to the upper side thereof to fix the entire nose ridge reshaping device 10 with respect to the face such that the nose ridge reshaping piece 30 is held along the ridge of the nose. In this embodiment, however, the adhesive fixing piece 20 also has a function of making inconspicuous and correcting the wrinkles between both brows and on the forehead.

In other words, the forehead-wrinkle eliminating member 21 of the adhesive fixing piece 20 is formed so as to extend from between both brows laterally along the upper edges of the brows on the face, as shown in FIG. 1 and FIG. 3, and a pair of curved recesses 22, 22 are provided on the lower edges of the right and left portions of the main body 20a to bypass the brows. The forehead-wrinkle eliminating member 21, to be described later, is stuck on the face such that the wrinkles between the brows and on the forehead are stretched and corrected, being covered by the forehead-wrinkle eliminating member 21.

The nose ridge reshaping piece 30 is formed so as to extend with a defined width from the middle portion of the lower end of said adhesive fixing piece 20 directly downward along the ridge of the nose. As stated above, the nose ridge reshaping piece 30 comprises the plastic substrate sheet 11 which back is coated with the adhesive 12, such that the shape thereof is maintained when it is bent in the crosswise direction.

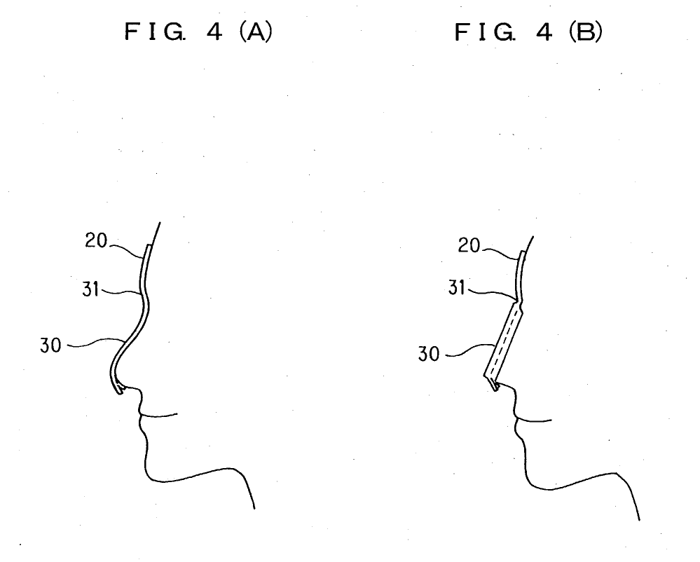

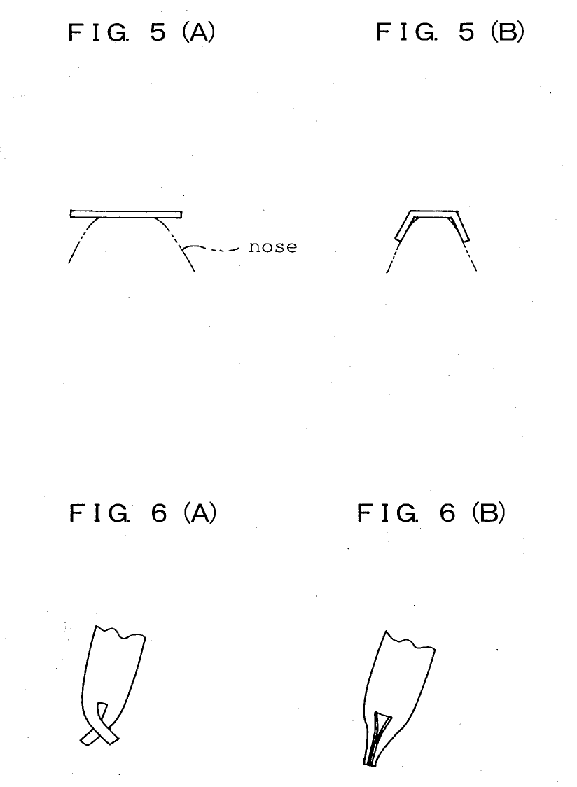

As shown in FIG. 4(A), the nose ridge reshaping piece 30 is deformably stuck from the upper end to the tip of the nose, being curved along the original ridge of the nose, with the cross section thereof being kept substantially linear (FIG. 5(A)), and then bent in the crosswise direction (FIG. 5(B)) such that the cross sectional surface thereof pinches the ridge of the nose, whereby the nose ridge reshaping piece 30 is held in the state of being extended linearly, with the ridge of the nose ranging from the upper end to the tip of the nose being raised forward, as shown in FIG. 4(B).

A fold origin 31 of the nose ridge reshaping piece 30 is provided on the border between the upper end of the nose ridge reshaping piece 30 and the lower end of the main body 20a of the adhesive fixing piece 20. The fold origin 31 provides a portion where the nose ridge reshaping piece 30 can be easily folded from the adhesive fixing piece 20 when the entire nose ridge reshaping piece 30 is extended in the longitudinal direction. The fold origin 31 comprises a pair of cuts 31a, 31a provided on the sides of the upper end of the nose ridge reshaping piece 30, the cuts 31a, 31a being adapted to produce a fold line 31b between them.

The lower end of the nose ridge reshaping piece 30 is provided with a hook terminal 32 that can be stuck to the tip of the nose so as to enclose it. The hook terminal 32 provides a portion for hooking the lower end of the nose ridge reshaping piece 30 to the tip of the nose. The hook terminal 32 comprises a pair of sticking pieces 32a, 32a produced by cutting out the middle portion of the lower edge, the sticking pieces 32a being adapted to wrap the tip of the nose when stuck together into a form as shown in FIG. 6(A) or FIG. 6(B).

Next, the usage and function of the nose ridge reshaping device 10 will be described.

When the nose ridge reshaping device is used, the forehead-wrinkle eliminating member 21 is assembled with the main body 20a of the adhesive fixing piece 20 such that they cross each other at substantially right angles, as shown in FIG. 1. Then, as shown in FIG. 3, the adhesive fixing piece 20a is first stuck on the face area ranging from between both brows to the upper side thereof, and the forehead-wrinkle eliminating member 21 is stuck to the forehead so as to extend laterally along the upper edges of the brows.

Because the forehead-wrinkle eliminating member 21 is stuck so as to stretch the wrinkles between the brows and on the forehead covered by the forehead-wrinkle eliminating member 21, the wrinkles in the area are held in the state of being stretched. Therefore, the wrinkles remain inconspicuous and milder even after the nose ridge reshaping device 10 is peeled off the face.

After the entire nose ridge reshaping device 10 is fixed to the face by sticking the adhesive fixing piece 20 between both brows and on the forehead, the nose ridge reshaping piece 30 extending from the lower end of the main body 20a directly downward is stuck along the ridge of the nose. As shown in FIG. 5(A), the nose ridge reshaping piece 30 can be easily curved when the cross section thereof is kept substantially linear. The nose ridge reshaping piece 30 in such a form is first stuck from the upper end of the ridge of the nose to the tip of the nose, with a natural curve being created along the original ridge of the nose, as shown in FIG. 4(A).

The lower end of the nose ridge reshaping piece 30 is provided with the hook terminal 32. The lower end of the nose ridge reshaping piece 30 can be firmly fixed to the tip of the nose by sticking the pair of sticking pieces 32a, 32a together into a form as shown in FIG. 6(A) or FIG. 6(B) so as to wrap the tip of the nose.

After that, the nose ridge reshaping piece 30 is bent in the crosswise direction such that the cross sectional surface thereof pinches the ridge of the nose, as shown in FIG. 5(B). The nose ridge reshaping piece 30 is bent into a substantially Π shape in this example but may be bent into a V or U shape. As a result, the nose ridge reshaping piece 30 is deformed from the state of being curved along the ridge of the nose into the state of being extended linearly in the longitudinal direction, as shown in FIG. 4(B).

Because the fold origin 31 of the nose ridge reshaping piece 30 is provided on the border between the upper end of the nose ridge reshaping piece 30 and the lower end of the main body 20a of the adhesive fixing piece 20, it is easy to fold the nose ridge reshaping piece 30 from the adhesive fixing piece 20 when the entire nose ridge reshaping piece 30 is extended in the longitudinal direction, and the adhesive fixing piece 20 is prevented from being subjected to an undesired peeling force.

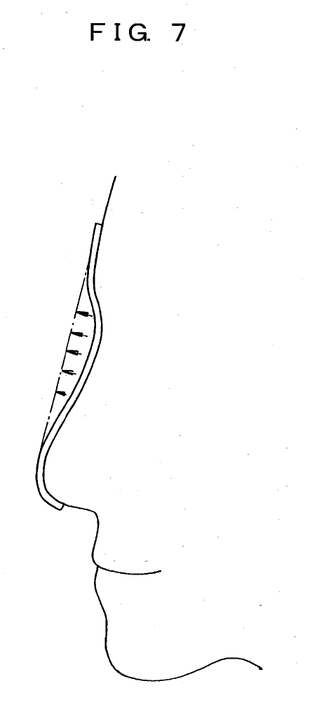

As a result of such deformation of the nose ridge reshaping piece 30, the ridge of the nose ranging from between both brows to the tip of the nose to which the nose ridge reshaping piece 30 is stuck is held in the state of being raised forward, as shown in FIG. 7. Thus, a clear straightforwardly extending ridge of nose which gives an attractive feeling can be provided.

The above nose ridge reshaping device 10 can effectively and easily correct the ridge of the nose and the wrinkles at the same time, being stuck on the face while sleeping at night, for example. Further, the nose ridge reshaping device 10 can be manufactured extremely easily and commercialized at a relatively low cost.

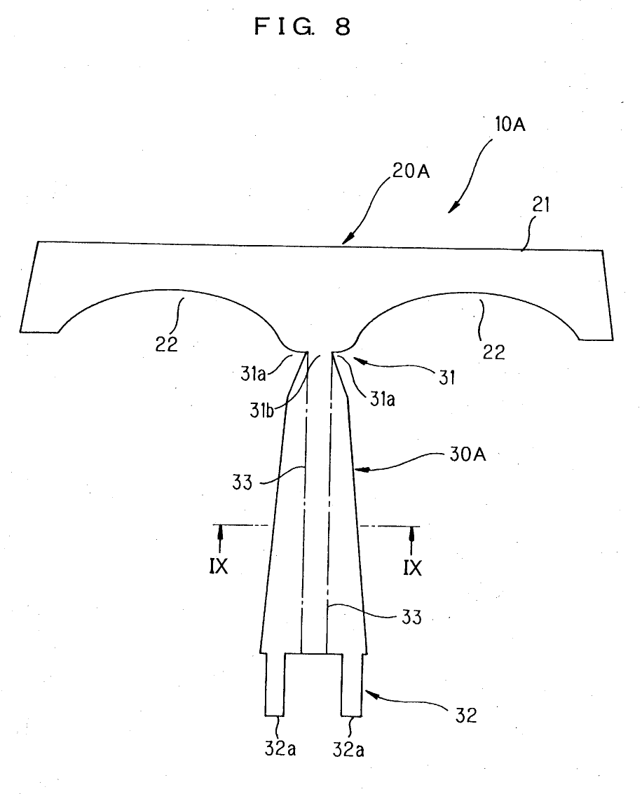

FIG. 8 to FIG. 10(B) show a second embodiment of the present invention.

The nose ridge reshaping device 10A according to the second embodiment has the same basic structure and function as those of the nose ridge reshaping device 10 according to the first embodiment. In the nose ridge reshaping device 10A according to the second embodiment, however, the adhesive fixing piece 20A and the nose ridge reshaping piece 30A are integral with each other across said fold origin 31, forming a substantially T shape as a whole, as shown in FIG. 8. The portions similar to those of the first embodiment are provided with the same reference characters to avoid duplication of explanations.

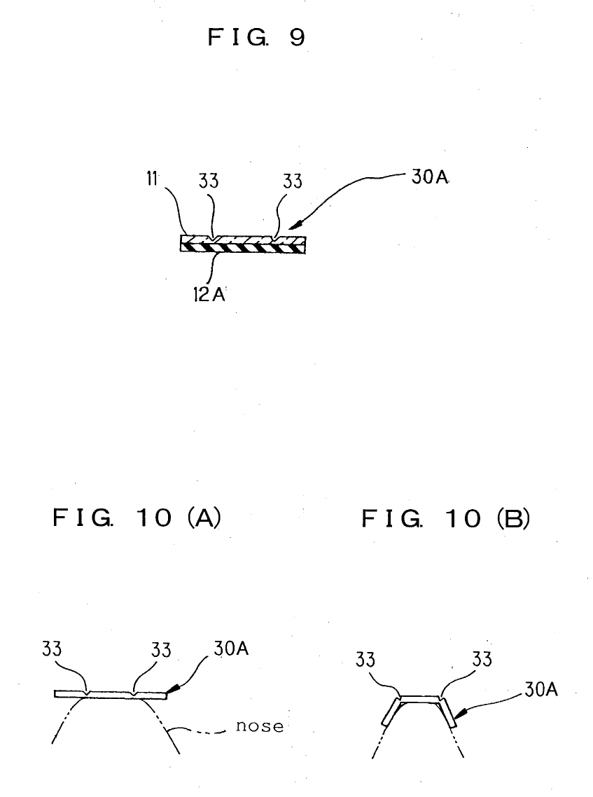

As shown in FIG. 9, the nose ridge reshaping piece 30A comprises a substrate sheet 11 which back is laminated with a pressure sensitive adhesive sheet 12A, which has been prepared in a sheet form beforehand. Preferably, the pressure sensitive adhesive sheet 12A also remains covered by a backing material (not shown) until it is used, in order to retain adhesion capability. This also applies to the adhesive fixing piece 20A.

If the pressure sensitive adhesive sheet 12A is replaceable with a new pressure sensitive adhesive sheet 12A, the substrate sheet 11 can be repeatedly used by merely replacing the pressure sensitive adhesive sheet 12A whose adhesion capability has been decreased, which can lead to a reduction in cost. For example, a commercially available medical tape may be used as the pressure sensitive adhesive sheet 12A.

As shown in FIG. 8, the nose ridge reshaping piece 30A is formed integrally with the adhesive fixing piece 20A such that the nose ridge reshaping piece 30A extends from the middle portion of the lower end of the adhesive fixing piece 20A downward along the ridge of the nose, being provided with a defined width that slightly increases toward the lower end. Further, the nose ridge reshaping piece 30A is provided with a pair of fold grooves 33, 33 extending in the longitudinal direction and substantially parallel to each other such that the nose ridge reshaping piece 30A is easily bent in the crosswise direction.

With the above nose ridge reshaping device 10A according to this embodiment, the entire nose ridge reshaping device 10A is fixed onto the face by sticking the adhesive fixing piece 20A to the forehead, and then the nose ridge reshaping piece 30A extending from a substantially middle portion of the lower end of the adhesive fixing piece 20A directly downward is stuck along the ridge of the nose. As shown in FIG. 10(A), the nose ridge reshaping piece 30A can be easily curved when the cross sectional surface thereof is substantially linear. The nose ridge reshaping piece 30A in such a form is stuck from the upper end of the ridge of the nose to the tip of the nose, with a natural curve being created along the original ridge of the nose (see FIG. 4(A)).

Then, the nose ridge reshaping piece 30A is bent in the crosswise direction such that the cross sectional surface pinches the ridge of the nose, as shown in FIG. 10(B). The nose ridge reshaping piece 30A is provided with a pair of fold grooves 33, 33 extending in the longitudinal direction and substantially parallel to each other, and can be easily bent at fold grooves 33 such that the cross sectional surface thereof forms a substantially Π shape.

When the nose ridge reshaping piece 30A is bent in the crosswise direction as stated above, the longitudinal section is plastically deformed from the state of being curved along the ridge of the nose into the state of being extended substantially linearly (see FIG. 4(B)). Because the ridge of the nose to which the nose ridge reshaping piece 30A is stuck is held in the state of being raised forward (see FIG. 7), a clear straightforwardly extending ridge of nose which gives an attractive feeling can be provided.

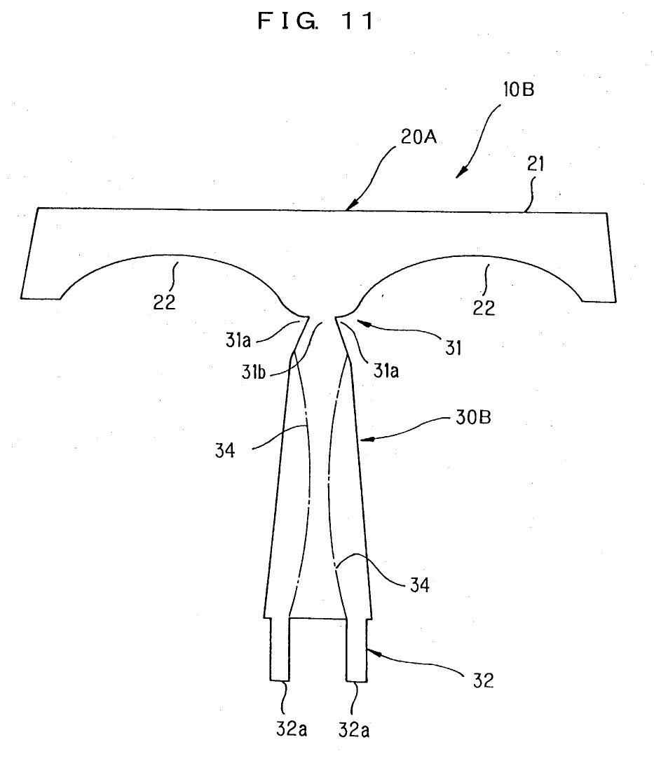

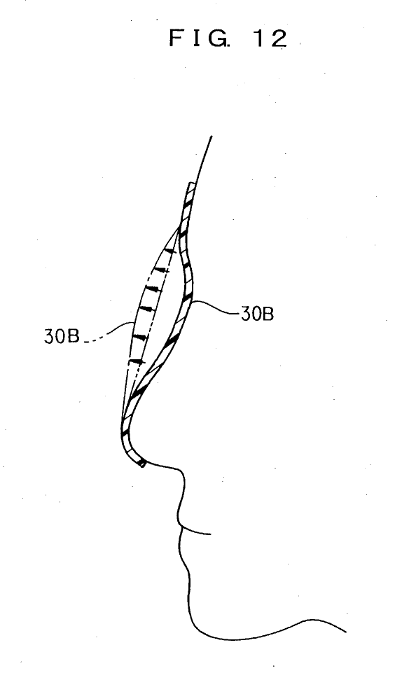

FIG. 11 and FIG. 12 show a third embodiment of the present invention.

In the nose ridge reshaping device 10B according to the third embodiment, the nose ridge reshaping piece 30B is provided with a pair of fold grooves 34, 34 extending in the longitudinal direction and forming curves that approach each other at substantially middle points, as shown in FIG. 11. The portions similar to those of the second embodiment are provided with the same reference characters to avoid duplication of explanations.

According to such curved fold grooves 34, when the nose ridge reshaping piece 30B is bent in the crosswise direction at said fold grooves 34 so as to pinch the ridge of the nose, the nose ridge reshaping piece 30B is plastically deformed into and held in the state of raising the ridge of the nose forward, with the longitudinal sectional surface thereof being extended forward with a slight curve, as shown in FIG. 12.

Because the nose ridge reshaping piece 30B is extended forward with the longitudinal sectional surface thereof being slightly curved, rather than being held substantially linearly, the ridge of the nose is raised forward even further by the curve. Thus, a still clear straightforwardly extending ridge of nose can be provided.

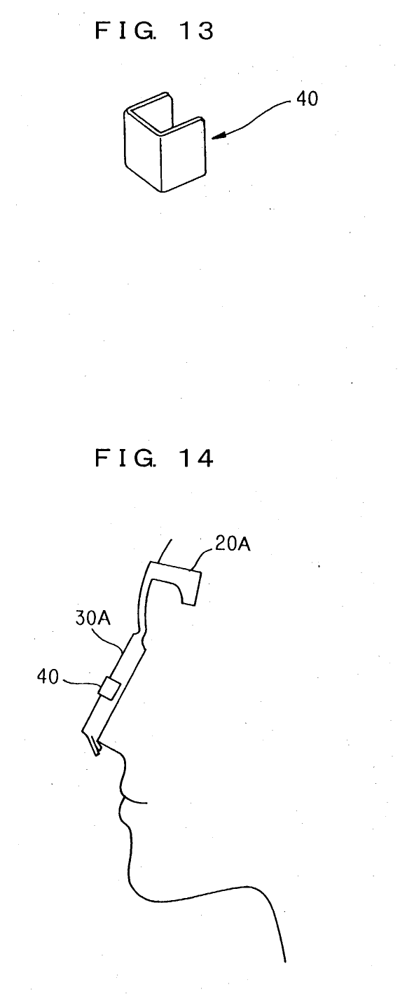

FIG. 13 and FIG. 14 show a fourth embodiment of the present invention.

In the fourth embodiment, a holder 40 such as that shown in FIG. 13 is used in combination. The holder 40 can be attached to the bent nose ridge reshaping piece 30A so as to pinch it at an intermediate point when said nose ridge reshaping piece 30A is bent in the crosswise direction and may be made of such materials as elastic synthetic resin such that the cross sectional surface forms a substantially Π shape.

As shown in FIG. 14, because such holder 40 is attached to the bent nose ridge reshaping piece 30A so as to pinch it at an intermediate point when said nose ridge reshaping piece 30A is bent in the crosswise direction, the bent geometry can be secured for a long period of time. Naturally, the holder 40 may be combined with the nose ridge reshaping piece 30 or the nose ridge reshaping piece 30B.

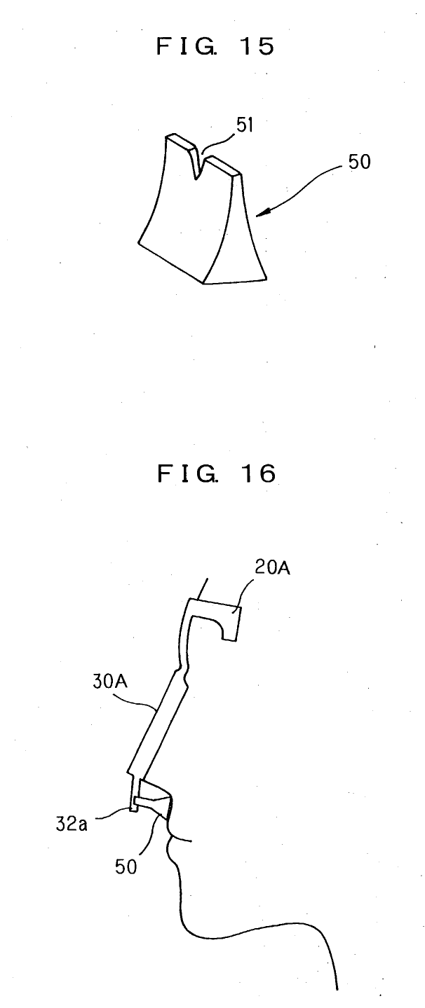

FIG. 15 and FIG. 16 show a fifth embodiment of the present invention.

In the fifth embodiment, a support 50 such as that shown in FIG. 15 is used in combination. The support 50 is interposed between the hook terminal 32 and the bottom of the nose such that the sticking pieces 32a of the hook terminal 32 extending over the bottom of the nose are raised forward when said hook terminal 32 encloses the tip of the nose.

For example, the support 50 is made of such a material as synthetic resin in a shape as shown in FIG. 15 and is provided with a recess 51 at an end for engaging the pair of sticking pieces 32a, 32a that have been stuck together.

As shown in FIG. 16, such support 50 is interposed between the hook terminal 32 and the bottom of the nose such that the sticking pieces 32a of the hook terminal 32 are raised forward when said hook terminal 32 encloses the tip of the nose. As a result, the tip of the nose is prevented from being pressed and crushed by said nose ridge reshaping piece 30A in reaction to the longitudinal extension of said nose ridge reshaping piece 30A, allowing the entire nose, including the tip, to be raised forward securely. Naturally, the support 50 may be combined with the nose ridge reshaping piece 30 or the nose ridge reshaping piece 30B.

Although some embodiments of the present invention have been described with reference to the drawings, it should be noted that specific arrangement is not limited to those described in the embodiments, and any modification or addition within the scope of the subject matters of the present invention is included in the present invention. For example, the specific forms of the adhesive fixing piece 20, 20A and the nose ridge reshaping piece 30, 30A, 30B are not limited to those provided in the drawings.

Further, said adhesive fixing piece 20, 20A may be formed so as to extend laterally in a curve to fit the forehead, rather than in a simple plane. In this case, said adhesive fixing piece 20, 20A is prevented from excessively pulling the skin in the vicinity of hairline when it is stuck to the forehead, and the possibility of inhibiting hair growth is eliminated.

Further, the areas on the nose ridge reshaping piece 30A, 30B to be laminated with the pressure sensitive adhesive sheet 12A may be limited to the narrow area between the fold grooves 33, 34 and the back of the sticking pieces 32a. For the lateral areas on the nose ridge reshaping piece 30A, 30B located outside the fold grooves 33, 34, the pressure sensitive adhesive sheet 12A may be omitted, or a nourishing cream, pack agent with cosmetic effect, or the like may be applied in place of the pressure sensitive adhesive sheet 12A.

The nose ridge reshaping device according to the present invention allows plastically reshaping a nose into a clear straightforwardly extending shape, correcting the ridge of the nose to an attractive feeling, by sticking an adhesive fixing piece on the area ranging from between both brows to the upper side thereof to fix the entire nose ridge reshaping device because the ridge of the nose ranging from between both brows to the tip of the nose to which the nose ridge reshaping piece is stuck is held in the state of being raised forward.

Further, said nose ridge reshaping piece, which is bent in the crosswise direction such that the cross sectional surface thereof pinches the ridge of the nose, is provided with a pair of fold grooves extending in the longitudinal direction and thus can be easily bent at the fold grooves such that the cross sectional surface thereof forms a substantially Π shape.

Also, wrinkles can be reduced or made inconspicuous by forming said adhesive fixing piece so as to extend from between both brows laterally along the upper edges of the brows and sticking it to the face so as to stretch and correct the wrinkles in the area covered by the adhesive fixing piece.

What is claimed is:

1. A nose ridge reshaping device (10) allowing reshaping of the ridge of a nose into a clear straightforwardly extending shape by sticking it on a face, comprising:
an adhesive fixing piece (20) to be stuck on the area ranging from between both brows to the forehead; and
a nose ridge reshaping piece (30) extending from the adhesive fixing piece (20) downward with a defined width that is plastic and can be curved along the ridge of the nose,
wherein said nose ridge reshaping piece (30) can be deformed in the longitudinal direction, with the cross sectional surface thereof being held substantially linearly, and is stuck from said adhesive fixing piece (20) to the tip of the nose along the original ridge of the nose and then bent in the crosswise direction such that the cross sectional surface thereof pinches the ridge of the nose, whereby the longitudinal sectional surface thereof is held in the state of being extended substantially linearly, with the ridge of the nose ranging from the upper end of the nose ridge reshaping piece (30) to the tip of the nose being raised forward.

2. A nose ridge reshaping device (10A) allowing reshaping of the ridge of a nose into a clear straightforwardly extending shape by sticking it on a face, comprising:
an adhesive fixing piece (20A) to be stuck on the area ranging from between both brows to the forehead; and
a nose ridge reshaping piece (30A) extending from the adhesive fixing piece (20A) downward with a defined width that is plastic and can be curved along the ridge of the nose,
wherein said nose ridge reshaping piece (30A) is provided with a pair of fold grooves (33, 33) extending in the longitudinal direction and substantially parallel to each other,
said nose ridge reshaping piece (30A) being stuck from said adhesive fixing piece (20A) to the tip of the nose along the original ridge of the nose, being deformable in the longitudinal direction, with the cross sectional surface thereof being held substantially linearly, and then bent at said pair of fold grooves (33, 33) in the crosswise direction such that the cross sectional surface thereof pinches the ridge of the nose, whereby the longitudinal sectional surface is held in the state of being extended substantially linearly, with the ridge of the nose being raised forward by the nose ridge reshaping piece (30A).

3. A nose ridge reshaping device (10B) allowing reshaping of the ridge of a nose into a clear straightforwardly extending shape by sticking it on a face, comprising:
an adhesive fixing piece (20A) to be stuck on the area ranging from between both brows to the forehead; and a nose ridge reshaping piece (30B) extending from the adhesive fixing piece (20A) downward with a defined width that is plastic and can be curved along the ridge of the nose,
wherein said nose ridge reshaping piece (30B) is provided with a pair of fold grooves (34, 34) extending in the longitudinal direction and forming curves that approach each other at substantially middle points,
said nose ridge reshaping piece (30B) being stuck from said adhesive fixing piece (20A) to the tip of the nose along the original ridge of the nose, being deformable in the longitudinal direction, with the cross sectional surface thereof being held substantially linearly, and then bent at said pair of fold grooves (34, 34) in the crosswise direction such that the cross sectional surface thereof pinches the ridge of the nose, whereby the longitudinal sectional surface thereof is held in the state of being extended forward with a slight curve, with the ridge of the nose being raised forward by the nose ridge reshaping piece (30B).

4. The nose ridge reshaping device(10, 10A, 10B) according to claim 1, 2 or 3, wherein each of said adhesive fixing piece (20, 20A) and nose ridge reshaping piece (30, 30A, 30B) comprises a plastic substrate sheet (11) which back is coated with an adhesive (12).

5. The nose ridge reshaping device (10, 10A, 10B) according to claim 1, 2 or 3, wherein each of said adhesive fixing piece (20, 20A) and nose ridge reshaping piece (30, 30A, 30B) comprises a plastic substrate sheet (11) which back is laminated with a pressure sensitive adhesive sheet (12A), the pressure sensitive adhesive sheet (12A) being replaceable with a new pressure sensitive adhesive sheet (12A).

6. The nose ridge reshaping device (10, 10A, 10B) according to claim 1, 2, 3, 4 or 5, further comprising:
a holder (40) that can be attached to the bent nose ridge reshaping piece (30, 30A, 30B) so as to pinch it at an intermediate point when said nose ridge reshaping piece (30, 30A, 30B) is bent in the crosswise direction, in order to hold said bent geometry.

7. The nose ridge reshaping device (10, 10A, 10B) according to claim 1, 2, 3, 4, 5 or 6, wherein the lower end of said nose ridge reshaping piece (30, 30A, 30B) is provided with a hook terminal (32) that can be stuck to the tip of the nose so as to enclose it.

8. The nose ridge reshaping device (10, 10A, 10B) according to claim 7, further comprising:
a support (50) that is interposed between said hook terminal (32) and the bottom of the nose such that the hook terminal (32) is raised forward when the hook terminal (32) encloses the tip of the nose.

9. The nose ridge reshaping device (10, 10A, 10B) according to claim 1, 2, 3, 4, 5, 6, 7 or 8, wherein a fold origin (31) of the nose ridge reshaping piece (30, 30A, 30B) is provided on the border between the upper end of said nose ridge reshaping piece (30, 30A, 30B) and the lower end of said adhesive fixing piece (20, 20A).

10. The nose ridge reshaping device (10, 10A, 10B) according to claim 1, 2, 3, 4, 5, 6, 7, 8 or 9, wherein at least a part of said adhesive fixing piece (20, 20A) and said nose ridge reshaping piece (30, 30A, 30B) are formed separately and combined when used.

11. The nose ridge reshaping device (10, 10A, 10B) according to claim 1, 2, 3, 4, 5, 6, 7, 8, 9 or 10, wherein said adhesive fixing piece (20, 20A) is formed so as to extend from between both brows laterally along the upper edges of the brows and is stuck so as to stretch and correct the wrinkles in the area covered by the adhesive fixing piece (20, 20A).

12. The nose ridge reshaping device (10, 10A, 10B) according to claim 11, wherein said adhesive fixing piece (20, 20A) is formed so as to extend laterally in a curve to fit the forehead.
